(19) United States
(12) Patent Application Publication
(10) Pub. No.: US 2025/0049364 A1
(43) Pub. Date: Feb. 13, 2025

(54) Noninvasive biomarker for diagnosing major depressive disorder using optical coherence tomography

(71) Applicant: Anvitha Narasimha Addanki, Canton, MA (US)

(72) Inventor: Anvitha Narasimha Addanki, Canton, MA (US)

(21) Appl. No.: 18/447,909

(22) Filed: Aug. 10, 2023

(51) Int. Cl. A61B 5/16; A61B 3/10; A61B 5/107; G16H 50/20

(52) U.S. Cl. CPC A61B 5/165; A61B 3/102; A61B 5/1075; A61B 5/1079; G16H 50/20

(57) ABSTRACT

Major depressive disorder (MDD) is a common mental disorder that affects adolescents and adults, causing staggering economic burdens, disabilities in the workforce, and suicidal thoughts, if not treated in time. In one or more implementations, a combination of Retinal Nerve Layer thickness measurements for a subject may be selected from a plurality of features, and a machine learning model may be trained to predict if the subject has MDD or is at risk for developing MDD by providing a prediction score. Machine learning models may additionally be configured to predict subtypes of MDD. Multiple machine learning models and algorithms are periodically trained, validated, and tested to diagnose MDD, and performance is evaluated for each trained model using evaluation metrics such as Accuracy, Precision, Sensitivity, and Specificity. The best model, which provides the highest Accuracy and Sensitivity to diagnose MDD, is redeployed periodically.

### RELATED APPLICATIONS

The present disclosure relates to Chinese Patent Application No: CN102046067B, titled “Optical coherence tomography device, method and system,” Publication Date: Oct. 31, 2017, and hereby incorporated by reference herein in their entirety.

The present disclosure relates to International Patent Application No: PCT/US2009/051073, titled “OPTICAL COHERENCE TOMOGRAPHY-BASED OPHTHALMIC TESTING SYSTEMS,” now International Publication No.: WO 2010/009447 A2, International Publication Date: Jan. 21, 2010, and hereby incorporated by reference herein in their entirety.

The present disclosure relates to International Patent Application No. PCT/US2019/015036, titled “OPTICAL COHERENCE TOMOGRAPHY IMAGING SYSTEMS, HANDHELD PROBES, AND METHODS THAT USE A FIELD CURVATURE TO MATCH A CURVED SURFACE OF TISSUE,” now International Publication No.: WO 2019/147871 A1, International Publication Date: Aug. 1, 2019, and hereby incorporated by reference herein in their entirety.

The present disclosure relates to U.S. patent application Ser. No. 17/475,153, titled “OPTICAL COHERENCE TOMOGRAPHY-BASED OPHTHALMIC TESTING METHODS, DEVICES AND SYSTEMS,” now U.S. Pat. No. 11,510,567 B2, Date of Patent: Nov. 29, 2022, and hereby incorporated by reference herein in their entirety.

### CROSS-REFERENCES

Jindahra, Petrie, Aviva, & T., G. (2012, Feb. 1). Time course of retrograde trans-synaptic degeneration following occipital lobe damage in humans. Retrieved Dec. 16, 2018, from https://academic.oup.com/brain/article/135/2/534/262691.

Elvina May-Yin Chu, A. (2012, Jul. 30). A window into the brain: An in vivo study of the retina in schizophrenia using optical coherence tomography. Retrieved Dec. 14, 2018, from https://www.ncbi.nlm.nih.gov/pmc/articles/PMC4024658/.

Kromer, Robert, Serbecic, Nermin, Hausner, Fahmy, Sven. (2014, Feb. 11). Detection of Retinal Nerve Fiber Layer Defects in Alzheimer's Disease Using SD-OCT. Retrieved Dec. 18, 2018, from https://www.frontiersin.org/articles/10.3389/fpsyt.2014.00022/full.

Kim, M., Kwon, J. W., Kim, J. W., & Kim, D. M. (2011, Aug. 1). Retinal Nerve Fiber Layer Defect and Cerebral Small Vessel Disease. Retrieved Dec. 18, 2018, from https://iovs.arvojournals.org/article.aspx?articleid=2188375.

Kelsey L. Thomson, K. (2015, Jun. 1). A systematic review and meta-analysis of retinal nerve fiber layer change in dementia, using optical coherence tomography. Retrieved Dec. 14, 2018, from https://www.sciencedirect.com/science/article/pii/S2352872915000366.

Glover, G. H. (2011, April). Overview of functional magnetic resonance imaging. Neurosurgery clinics of North America. Retrieved Dec. 20, 2018, from https://www.ncbi.nlm.nih.gov/pmc/articles/PMC3073717/.

Dickerson, B. C. (n.d.). Advances in functional magnetic resonance imaging: Technology and clinical applications. Neurotherapeutics: the journal of the American Society for Experimental NeuroTherapeutics. Retrieved Dec. 20, 2018, from https://pubmed.ncbi.nlm.nih.gov/17599702/.

Kango, A., Grover, S., Gupta, V., Sahoo, S., & Nehra, R. (2022, Dec. 7). A comparative study of retinal layer changes among patients with schizophrenia and healthy controls: Acta Neuropsychiatrica. Cambridge Core. Retrieved Dec. 21, 2018, from https://www.cambridge.org/core/journals/acta-neuropsychiatrica/article/abs/comparative-study-of-retinal-layer-changes-among-patients-with-schizophrenia-and-healthy-controls/D813C2EE5191EDEFDC271C5E20670061.

Johnson, J. (2021). Duct leakage detection system and method (U.S. Patent No. US 20190025154A1). U.S. Patent and Trademark Office. Retrieved Dec. 23, 2018, from https://patents.google.com/patent/US20190025154A1/.

Huang, D., Liu, G., Jia, Y. (2023). Systems and methods for automated widefield optical coherence tomography angiography (U.S. Patent No. US20230069484A1). U.S. Patent and Trademark Office. Retrieved Dec. 23, 2018, from https://patents.google.com/patent/US20230069484A1.

### REFERENCE TO RELATED PATENTS

The present disclosure relates to U.S. patent application Ser. No. 13/294,601, titled “AUTOMATED MACULAR PATHOLOGY DIAGNOSIS IN THREE-DIMENSIONAL (3D) SPECTRAL DOMAIN OPTICAL COHERENCE TOMOGRAPHY (SD-OCT) IMAGES,” now U.S. Pat. No. 8,712,505 B2, Date of Patent: Apr. 29, 2014, and hereby incorporated by reference herein in their entirety.

The present disclosure relates to U.S. patent application Ser. No. 15/253,597, titled “SYSTEMS AND METHODS OF GLAUCOMA DIAGNOSIS BASED ON FREQUENCY ANALYSIS OF INNER RETINAL SURFACE PROFILE MEASURED BY OPTICAL COHERENCE TOMOGRAPHY,” now U.S. Pat. No. 9,918,630 B2, Date of Patent: Mar. 20, 2018, and hereby incorporated by reference herein in their entirety.

The present disclosure relates to International Patent Application No. PCT/JP2021/023628, titled “MEDICAL DIAGNOSTIC APPARATUS AND METHOD FOR EVALUATION OF PATHOLOGICAL CONDITIONS USING 3D OPTICAL COHERENCE TOMOGRAPHY DATA AND IMAGES,” now International Publication No.: WO 2022/004492 A1, International Publication Date: Jan. 6, 2022, and hereby incorporated by reference herein in their entirety.

### BACKGROUND OF THE INVENTION

The present invention relates to identifying a novel, noninvasive and inexpensive biomarker for diagnosing Major Depressive Disorder (MDD) using Optical Coherence Tomography (OCT) scans. This invention will allow for early diagnosis and treatment of MDD in humans and can improve their overall health and quality of life.

Major Depressive Disorder (MDD) is one of the most common mental disorders and causes a persistent feeling of sadness and loss of interest. It affects how people feel, think, and behave and can lead to various emotional and physical problems. People experience decreased quality of life and may have trouble doing normal day-to-day activities, and sometimes may feel as if life is not worth living, leading to suicides.

Findings from past studies indicate that patients with MDD have negative cognitive disorder and extensive brain dysfunction, potentially caused by reduced activation of the Occipital Lobe. Researchers found that occipital bending is three (3) times more common among patients with MDD than healthy subjects.

The Optical Coherence Tomography (OCT) apparatus is a noninvasive imaging technology used to obtain high-resolution cross-sectional images of biological tissues, such as the retina. It works on the principle of low-coherence interferometry, where light is split into two beams, one of which is directed towards the tissue being imaged and the other towards a reference mirror. The backscattered light from the tissue and reference mirror is combined, and interference patterns are created that can be analyzed to obtain information about the thickness and structure of the tissue.

In the case of the Retinal Nerve Fiber Layer (RNFL), which is a thin layer of nerve fibers that connects the retina to the brain, the OCT apparatus can provide precise measurements of the RNFL thickness. The RNFL thickness is an important clinical parameter that can be used to diagnose and monitor the progression of various ocular diseases, such as glaucoma, which can cause thinning of the RNFL.

During an OCT scan, the patient's eye is typically dilated with eye drops, and the OCT apparatus is positioned in front of the eye. The apparatus scans the retina with a focused beam of light and generates a cross-sectional image of the retina, including the Retinal Nerve Fiber Layer (RNFL), Ganglion Cell Layer-Inner Plexiform Layer (GCL-IPL), and other retinal layers. The thickness of the RNFL, GCL-IPL, and other retinal layers can be measured at various locations around the optic nerve head, and these measurements can be used to detect any thinning or abnormalities in the RNFL, GCL-IPL, and other retinal layers that may be indicative of ocular disease.

### DESCRIPTION

Embodiments of the present systems and methods may provide a noninvasive system to enable early detection of Major Depressive Disorder (MDD) and other neurological diseases and track their progression by measuring the Retinal Nerve Fiber Layer (RNFL), Ganglion Cell Layer-Inner Plexiform Layer (GCL-IPL), and other retinal layer thicknesses either independently or together and also by using various measurements of the gyri in the Occipital Lobe using functional Magnetic Resonance Imaging (fMRI) scans.

### BRIEF STATEMENT OF THE INVENTION

This invention provides methods of noninvasively diagnosing Major Depressive Disorder (MDD) using Optical Coherence Tomography (OCT) apparatus and measuring various attributes of the Retinal Nerve Fiber Layer (RNFL), Ganglion Cell Layer-Inner Plexiform Layer (GCL-IPL), and other retinal layers such as thickness and other measures.

Neurological diseases such as Multiple Sclerosis (MS), Alzheimer's disease, Parkinson's disease, and others have been reported to affect the RNFL, GCL-IPL, and other retinal layer thicknesses. In one embodiment, the invention also provides methods of noninvasively diagnosing the above-mentioned neurological diseases using Optical Coherence Tomography (OCT) devices and measuring various attributes of RNFL, GCL-IPL, and other retinal layers such as thickness and other measures.

RNFL, GCL-IPL, and other retinal layers thickness can also provide information about the health of the optic nerve and visual pathways, which are essential for proper visual function, including the processing of visual information in the Occipital Lobe (FIG. 3). In one embodiment, the invention also involves in identifying the differences in the Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume of all the gyri in the Occipital Lobe. The differences are identified using corresponding mean measurements of healthy people of the same age and gender with machine learning models and artificial intelligence to noninvasively diagnose Major Depressive Disorder (MDD) and other neurological diseases mentioned above.

### SUMMARY OF THE INVENTION

Embodiments of the current invention would provide various automated methods, including machine learning models, artificial intelligence techniques, and traditional statistical analysis models, to enable early detection and monitoring of Major Depressive Disorder (MDD) and other neurological diseases mentioned above noninvasively.

Optical Coherence Tomography (OCT) uses low-coherence interferometry to create high-resolution, cross-sectional images of the retina. The patient sits in front of an OCT device (FIG. 4), and a light beam is scanned across the retina, creating a detailed 3D image of the different retinal layers. This image can then be analyzed to measure the thickness of the Retinal Nerve Fiber Layer (RNFL), Ganglion Cell Layer-Inner Plexiform Layer (GCL-IPL), and other retinal layers. OCT is a quick and painless procedure that requires no special preparation from the patient. The test takes only a few minutes to complete, and the results are available immediately.

Layer thickness can be measured in both eyes (Left Eye—OS—Oculus Sinister and right eye—OD—oculus dextrus) using average and individual quadrant measures: OD—Temporal (μm), OD—Superior (μm), OD—Nasal (μm), OD—Inferior (μm), OD—Average (μm), OS—Temporal (μm), OS—Superior (μm), OS—Nasal (μm), OS—Inferior (μm), and OS—Average (μm) using commercial OCT/OCTA devices. According to an aspect of the invention, automated methods are then executed using these measurements to predict Major Depressive Disorder (MDD) and other neurological diseases.

According to a further aspect of the invention, if functional Magnetic Resonance Imaging (fMRI) scans are available, this invention also provides automated methods to identify Major Depressive Disorder (MDD) and other neurological diseases using the various measurements of the gyri, including Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume of the Occipital Lobe using voxel analysis.

### OBJECT OF THE INVENTION

It is the object of this invention to improve the early diagnostic methods of Major Depressive Disorder (MDD) and other neurological diseases and their progression using noninvasive techniques such as OCT and other tests conducted periodically during routine medical visits. If fMRI scans are readily available, they can be used instead of OCT scans to achieve the same objectives.

### FIELD OF THE INVENTION

The present inventions relate to methods and systems for noninvasive early prediction and diagnosis of Major Depressive Disorder (MDD) and other neurological diseases and their progression with Optical Coherence Tomography (OCT) using machine learning and artificial intelligence techniques. More specifically, the field involves methods of detecting images and computing various attributes such as the thickness of various layers, surface areas, and volumes of different components in the eye and brain using OCT and fMRI images and various automated techniques to predict and diagnose MDD and other neurological diseases and monitor their progression.

### TECHNICAL FIELD

Generally, the field involves methods of using Optical Coherence Tomography (OCT) in the early detection and diagnosis of Major Depressive Disorder (MDD) and other neurological diseases and their progression. More specifically, the field involves methods of processing OCT images and using various attributes such as the thickness of various layers, surface areas, and volumes of different components in the eye and brain using OCT and fMRI images. Processing is done using various automated techniques such as machine learning models, artificial intelligence techniques, and traditional statistical analysis models to enable early prediction and monitoring of Major Depressive Disorder (MDD) and other neurological diseases noninvasively.

### PRIOR ART

Patent Document 1: United States Patent-Publication No.: US20140221780A1; Title: Diagnosis and monitoring of depression based on multiple biomarker panels; Publication Date: 2014 Aug. 7.
Patent Document 2: Worldwide Patent-Publication No.: WO2014144605A1; Title: Biomarkers for major depressive disorder; Publication Date: 2014 Sep. 18.
Patent Document 3: Japanese Patent-Publication No.: JP5663314B2; Title: Diagnosis and monitoring of depression based on multiple biomarker panels; Publication Date: 2015 Feb. 4.
Patent Document 4: Worldwide Patent-Publication No.: WO2015082927A1; Title: Novel biomarker panel for major depressive disease; Publication Date: 2015 Jun. 11.
Patent Document 5: United States Patent-Publication No.: US20160342757A1; Title: Diagnosing and monitoring depression disorders; Publication Date: 2016 Nov. 24.
Patent Document 6: United States Patent-Publication No.: US20170354363A1; Title: Diagnosing and monitoring depression disorders; Publication Date: 2018 Apr. 10.
Patent Document 7: United States Patent-Publication No.: US20220341945A1; Title: Biomarker for diagnosing depression and uses thereof; Publication Date: 2022 Oct. 27.

### DESCRIPTION OF THE PRIOR ART

Patent US20140221780A1 is directed to methods and systems for diagnosing depression and similar illnesses using complexity analysis of physiologic signals such as heart rate, voice/speech, and brain waves, which often need a separate laboratory or specialist doctor's appointment.

Patent WO2014144605A1 uses Complement Factor H Related Protein (CFHRP) as a biomarker for diagnosis of major depressive disorder, which involves obtaining biological samples from individuals in a medical laboratory and performing an extensive laboratory analysis for the analytes, ligands, and other bodily fluids within the samples.

Patent JP5663314B2 also involves obtaining a biological sample (e.g., a blood sample) from a depressed individual, measuring the level of a group of analytes such as brain-derived neurotrophic factor (BDNF), interleukin-7 (IL-7), and others in the sample, and determining an MDD disease score using an algorithm.

Patent WO2015082927A1 also involves obtaining a biological sample (e.g., blood and urine samples) from the patient, measuring the level of a group of analytes such as Interleukin-1 receptor antagonist (IL-Ira), Ferritin (FRTN), EN-RAGE and Tenascin-C (TNC), and others in the sample, and use them as biomarkers for the diagnosis of major depressive disorder, or predisposition to that.

Patent US20160342757A1 defines a disease score for a depression disorder (e.g., unipolar depression or major depressive disorder) in a subject using a multi-parameter system to measure a plurality of parameters and an algorithm to calculate the score.

Patent US20170354363A1 defines a system and a method for assessing MDD condition in a subject using phones from the speech of the subject.

Patent US20220341945A1 defines a method for diagnosing major depressive disorder by detecting the expression levels of ZA2G and prothrombin contained in the serum of the individuals.

### BRIEF STATEMENT OF THE PRIOR ART

Within the prior art, using physiologic signals such as heart rate, voice/speech, and phones from the speech and brain waves often needs a separate laboratory or specialist doctor's appointment. In addition, collecting biological samples from individuals requires a medical laboratory and an extensive laboratory analysis for identifying and quantifying the analytes, ligands, and other bodily fluids withos. It is inconvenient for individuals to provide these samples for laboratory analysis as they are not done as part of routine procedures and may be inaccessible due to high costs. Current solutions related to using speech analysis may be inaccurate and ineffective, as there may be significant variation between individuals and problems related to technology malfunctions.

### BACKGROUND ART

Optical coherence tomography (OCT) and optical coherence tomography angiography (OCTA) are noninvasive imaging tests. They use light waves to take cross-section pictures of the retina.

With OCT, ophthalmologists can see each of the retina's distinctive layers, which allows for mapping and measurement of their thicknesses. Currently, these measurements help with the diagnosis of various eye diseases such as glaucoma, retinal diseases like age-related macular degeneration (AMD), and diabetic eye disease.

Optical coherence tomography angiography (OCTA) takes pictures of the blood vessels in and under the retina. OCTA is similar to fluorescein angiography but is faster and does not use a dye.

To prepare the patient for an OCT exam, the ophthalmologist may or may not put dilating eye drops in their eyes. These drops widen the pupil and make it easier to examine the retina.

Patients will sit in front of the OCT machine and rest their heads on a support to keep it motionless. The equipment will then scan the eye without touching it. Scanning takes about 5 to 10 minutes. OCT test is conducted routinely in the ophthalmologist's office during routine vision checkups.

### DESCRIPTION OF RELATED ART

To address the challenges in current methods of diagnosing Major Depressive Disorder (MDD) which often takes a longer time, machine learning is used to predict MDD accurately with measurements extracted from Optical Coherence Tomography (OCT) scans, which are commonly available in hospitals and eye clinics.

There are currently very few conventional methods of diagnosing Major Depressive Disorder (MDD), such as using the PHQ-9 Depression Test Questionnaire. One of the major issues is the lack of clear biomarkers, as medical providers mainly depend on patient reports and observation. This may lead to misdiagnosis, delayed treatment, and lack of reliability in medical care. Additionally, MDD symptoms and severities may manifest differently among individuals, which creates difficulty in objective diagnosis. fMRI scans may also be used to analyze brain regions and determine activation levels in different areas, but these are often inconvenient, expensive, and time-consuming.

In one or more implementations, different combinations of features from measurements of Retinal Layer (FIG. 1) thickness may be selected from a plurality of features. These are based on metrics of performance and robustness of the combination, which can be used to train a machine learning model to predict Major Depressive Disorder (MDD) using the combination. The performance metric may be related to the Accuracy value of the machine learning models in predicting MDD, and the robustness metric may be related to the Sensitivity value of the machine learning models that can be related to the insensitivity of manufacturing variabilities on the accuracy of the Optical Coherence Tomography (OCT) devices. Upon training, the machine learning model can predict the likelihood of Major Depressive Disorder (MDD) for a subject using measurements of retinal layer thickness, demographic data (age, gender, ethnicity, and others), medical history (height, weight, body mass index, body fat, medical conditions), stress information, nutrition and exercise data, prescription history, occupation, and more for the related subjects. These combinations of features may be derived from the OCT measurements of the subject and are input into the machine learning model to predict MDD.

One or more machine learning models are developed using historical OCT data and historical MDD predictions of multiple subjects to predict Major Depressive Disorder (MDD) for a particular subject. OCT data and related measurements of the user population may be provided by eye doctors, manufacturers of OCT devices, and/or other members of the research community. Generally, historical outcome data incorporates diagnostic measures that are derived from sources independent of the OCT devices. For example, the historical MDD predictions may indicate whether a particular user is clinically diagnosed with MDD or not based on measurements of retinal layers using OCT imaging, therefore indicating an MDD classification that is clinically determined. In another example, another set of subjects may have been clinically diagnosed with MDD or not based on other conventional methods, which are independent of OCT measurements.

To determine the robustness metric and correlate the historic retinal layer measurements with MDD diagnoses, variance algorithms, such as Random Over-Sampling Examples (ROSE), Synthetic Minority Over-sampling Technique (SMOTE), and other methods may be utilized for over-sampling. Similarly, Edited Nearest Neighbors (ENN), Cluster Centroids, and other methods can be used for under-sampling. For instance, the robustness metric may be used to measure the average percentage change in the performance metric for every percent change in the simulated variance. The combination of features may be specifically selected to balance the performance and robustness metrics, such as selecting the combination of features that has the highest performance metric after removing candidate combinations that have a robustness metric below a certain threshold.

Once the combination of features is selected, multiple machine learning models are trained to predict the MDD classification based on these features, which are extracted from the historical Retinal Layer measurements, other subject-related data, and their related MDD outcome for the subject population, either with or without the simulated variance. After that, the trained machine learning models can process new Retinal Layer measurements provided by the OCT devices to predict the likelihood of MDD for any subject.

In one embodiment, trained machine learning models may predict the diagnosis of Major Depressive Disorder (MDD) for the subject.

In other embodiments, trained machine learning models may predict whether the subject is at risk for developing Major Depressive Disorder (MDD) by providing a prediction score, as well as guidance to the subject on the adverse effects associated with Major Depressive Disorder (MDD) they may experience.

In other embodiments, the machine learning model may alternatively be configured to predict a specific subtype of Major Depressive Disorder (MDD) such as Melancholic, Atypical, Psychotic, Seasonal Affective Disorder (SAD), or others.

In other embodiments, the machine learning model may additionally or alternatively be configured to predict a risk level of developing Major Depressive Disorder (MDD), such as high risk, low risk, or no risk for developing MDD.

In practice, Major Depressive Disorder (MDD) predicted by the machine learning models may include a detailed analysis of the prediction and can be used by the health care professionals to develop a treatment plan for the subject, similar to how the subject would be treated if clinically diagnosed using conventional methods.

### BRIEF DESCRIPTION OF FIGURES

FIG. 1 shows the retinal layers, which are crucial in the visual pathway. They include Retinal Nerve Fiber Layer (RNFL), Ganglion Cell Layer (GCL), Inner Plexiform Layer (IPL), Inner Nuclear Layer (INL), External Limiting Membrane (ELM), Outer Nuclear Layer (ONL), Outer Plexiform Layer (OPL), Inner limiting membrane (ILM), Retinal Pigment Epithelium (RPE), and Inner Segment/Outer Segment (IS/OS).

FIG. 2 shows the visual pathway from the retinal layers to Occipital Lobe, which is the route that visual information takes when moving from the eye to the Occipital Lobe (the primary visual processing area in the brain).

FIG. 3 shows the Occipital Lobe and its components, which are responsible for processing visual information. It is located at the back of the cerebral cortex, specifically in the posterior region. The Occipital Lobe plays a crucial role in visual perception, interpretation, and the formation of visual memories.

### BRIEF DESCRIPTION OF DRAWINGS

FIG. 4 shows the Optical Coherence Tomography (OCT) device, which is a noninvasive imaging tool used in ophthalmology and vision centers to visualize and capture detailed cross-sectional images of biological tissues, such as the retina, cornea, and other eye structures.

FIG. 5 shows the comparison of Cortical Mean Thickness, Inner Cortical Surface Area, Mid Cortical Surface Area, and Pial Cortical Surface Area of the Occipital Lobe, which is statistically significant (p_{Anova}<0.05) using voxel analysis for all age groups in multiple trials between Major Depressive Disorder (MDD) subjects and non-MDD subjects.

FIG. 6 shows the comparison of Grey Matter (GM) Volume, Cerebrospinal Fluid (CSF) Volume, White Matter (WM) Volume, and Total Volume of the Occipital Lobe, which is statistically significant (p_{Anova}<0.05) using voxel analysis for all age groups in multiple trials between Major Depressive Disorder (MDD) subjects and non-MDD subjects.

FIG. 7 shows the comparisons of Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume of depressed and non-depressed male and female subjects which is statistically significant (p_{Anova}<0.05).

FIG. 8 shows the central tendency, spread, and skewness of the data distribution of Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume of depressed and non-depressed subjects.

FIG. 9 shows the Retinal Nerve Fiber Layer (RNFL) measurements in all four quadrants for both eyes of a subject diagnosed with Major Depressive Disorder (MDD).

FIG. 10 shows the Ganglion Cell Layer (GCL) and Inner Plexiform Layer (IPL) measurements in all four quadrants for both eyes of a subject diagnosed with Major Depressive Disorder (MDD).

FIG. 11 shows the Inner limiting membrane (ILM) and Retinal Pigment Epithelium (RPE) measurements in all four quadrants for both eyes of a subject diagnosed with Major Depressive Disorder (MDD).

FIG. 12 shows the comparison of average Retinal Nerve Fiber Layer (RNFL) thickness in the left and right eye from OCT data which is statistically significant (p_{Anova}<0.05) for all age groups in multiple trials between Major Depressive Disorder (MDD) subjects and non-MDD subjects.

FIG. 13 shows the comparison of average Ganglion Cell Layer (GCL) and Inner Plexiform Layer (IPL) thickness in the left and right eye from OCT data, which is statistically significant (p_{Anova}<0.05) for all age groups in multiple trials between Major Depressive Disorder (MDD) subjects and non-MDD subjects.

FIG. 14 is a block diagram schematically illustrating one embodiment of the noninvasive biomarker system for diagnosing major depressive disorder using optical coherence tomography devices described herein.

FIG. 15 is a process flow diagram illustrating one embodiment of the noninvasive biomarker system for diagnosing major depressive disorder using optical coherence tomography devices described herein.

FIG. 16 is a process flow diagram illustrating one embodiment of the Data Digital Converter Module, which can read data from the OCT scan and related attributes in paper and other non-digital formats, extract the relevant OCT attributes, and convert them into a digital output.

FIG. 17 is a process flow diagram illustrating one embodiment of the Image Processing Module, which can preprocess images from the OCT scan, extract the relevant OCT attributes, and convert them into a digital output.

FIG. 18 is a process flow diagram illustrating one embodiment of the Machine Learning Model Manager 156, which is used to periodically train, validate, test, and manage multiple machine learning models to predict Major Depressive Disorder (MDD), by using OCT scan attributes, demographic data (age, gender, ethnicity, and others), medical history (height, weight, body mass index, body fat, medical conditions), stress information, nutrition and exercise data, prescription history, occupation, and more. Machine learning models include Decision Trees, Support Vector Machines, Random Forest Classifiers, Extreme Gradient Boosting, AdaBoost, Neural Networks, Multi-Layer Perceptron, and others. Machine Learning Model Manager also assesses the performance of each trained model using evaluation metrics such as Accuracy, Precision, Sensitivity, Specificity, and others. It selects the best model based on the performance criteria and deploys it for diagnosing Major Depressive Disorder (MDD). It also periodically trains all the models based on the newly available data and reselects the best model to diagnose Major Depressive Disorder (MDD).

FIG. 19 illustrates a block diagram of a computing system in accordance with some example embodiments.

FIG. 20 is a block diagram schematically illustrating one embodiment of how the noninvasive biomarker system for diagnosing major depressive disorder using optical coherence tomography devices described herein is used in practice. After the OCT scan is completed, OCT scan reports are automatically processed by the Data Processing Device, and an MDD probability score is generated almost instantly on both digital and non-digital formats.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that innovation can be practiced without these specific details. In other instances, well-known structures and devices are shown in block diagram form in order to facilitate describing the innovation.

As used in this application, the terms “component” and “system” are intended to refer to a computer-related entity (FIG. 19), either hardware, a combination of hardware and software, software, or software in execution. For example, a component can be but is not limited to a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

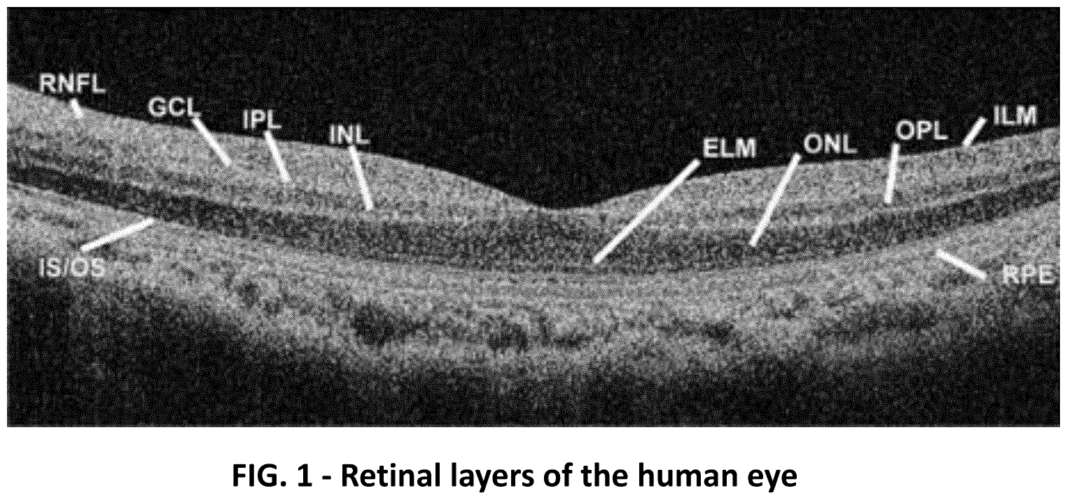

FIG. 1 illustrates various retinal layers of the human eye. Retinal layers are essential for the proper functioning of the retina, the structure that responds to visual information. These layers work together to process and transmit visual information from the environment to the brain via the optic nerve. Each layer plays a particular role in the process, ultimately contributing to the vision.

Treating various retinal diseases requires a thorough understanding of how the retinal layers are organized and function. For example, diseases that primarily affect the photoreceptor layer can result in impairment or loss of vision, while disorders involving the ganglion cell layer may cause damage to the optic nerve and defects in the corresponding visual fields. By evaluating the specific retinal layers that are affected, clinicians can develop targeted interventions and treatments to mitigate problems and preserve visual function.

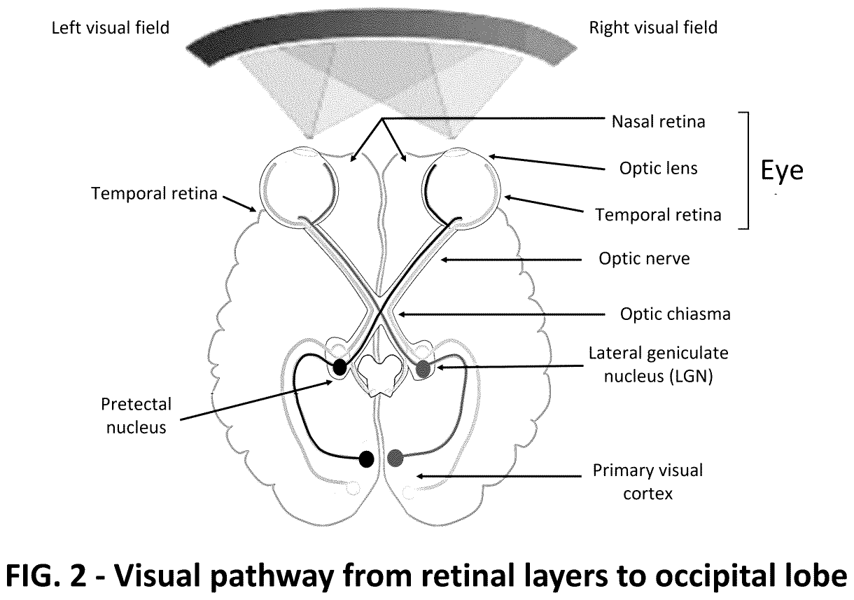

The retinal nerve layers are connected to the Occipital Lobe, which is the area in the brain that is responsible for processing visual information. This connection is mediated through the optic nerve, optic chiasm, optic tracts, and the visual pathway (FIG. 2).

Optic Nerve: The optic nerve is a bundle of fibers that carries visual information from the retina to the brain. Each optic nerve begins at the ganglion cells in the retina and exits the eye at the back of the eyeball. The optic nerves from both eyes converge at the base of the brain.

Optic Chiasm: The optic chiasm is a location at the base of the brain where the optic nerves partially intersect and cross over each other. This allows our visual system to integrate and process visual information coming from both eyes. The crossover results in the left side of the brain processing visual input from the right visual field and the right side of the brain processing visual input from the left visual field.

Optic Tracts: Upon reaching the optic chiasm, the nerve fibers continue propagating as optic tracts. These carry visual information that is at the optic chiasm to other brain structures for further processing, such as the lateral geniculate nucleus (LGN) of the thalamus.

Lateral Geniculate Nucleus (LGN): The LGN is a visual relay center within the thalamus that receives its input from the optic tracts. It serves as a primary processing station for visual information before it goes to the primary visual cortex in the Occipital Lobe.

Visual Cortex: The primary visual cortex is in the posterior part of the brain within the Occipital Lobe. It receives and further processes visual information to create perceptions such as color, form, motion, and depth. The cortex contains different areas within it that specialize in processing different aspects of visual inputs.

Transmission and processing of stimuli depend on connections between the retinal nerve layers and the Occipital Lobe. Visual information travels through neural pathways and undergoes complex analysis, which allows for visual perception.

The retina is the innermost layer in the eye and is responsible for converting light energy for photons into three-dimensional images. It is located in the posterior area of the eyeball and is the only extension of the brain that can be visualized from the outside. This provides a unique view into real-time pathology affecting the retina.

Retinal development begins during the fourth week of embryogenesis and continues into the first year of life. Thus, the retina is vulnerable to genetic and environmental changes that may affect its development. Retinal tissue consumes oxygen faster than any other tissue and is supplied by a dual bloodline that divides the retina into two portions for efficient delivery. The retina contains six different cell lines that are further differentiated into ten layers, each of which plays a unique role in processing visual information.

The retina contains ten different layers of neurons that are interconnected by synapses. The cells are further subdivided into three types: photoreceptor cells, neuronal cells, and glial cells. The layers from the closest to the front anterior of the head towards the posterior of the head are as follows: Retinal Nerve Fiber Layer (RNFL), Ganglion Cell Layer (GCL), Inner Plexiform Layer (IPL), Inner Nuclear Layer (INL), External Limiting Membrane (ELM), Outer Nuclear Layer (ONL), Outer Plexiform Layer (OPL), Inner limiting membrane (ILM), Retinal Pigment Epithelium (RPE), and Inner Segment/Outer Segment (IS/OS), and the layer of rods and cones. These layers contain different types of cells that all contribute to transmitting photons into action potentials to process light information into visual perception.

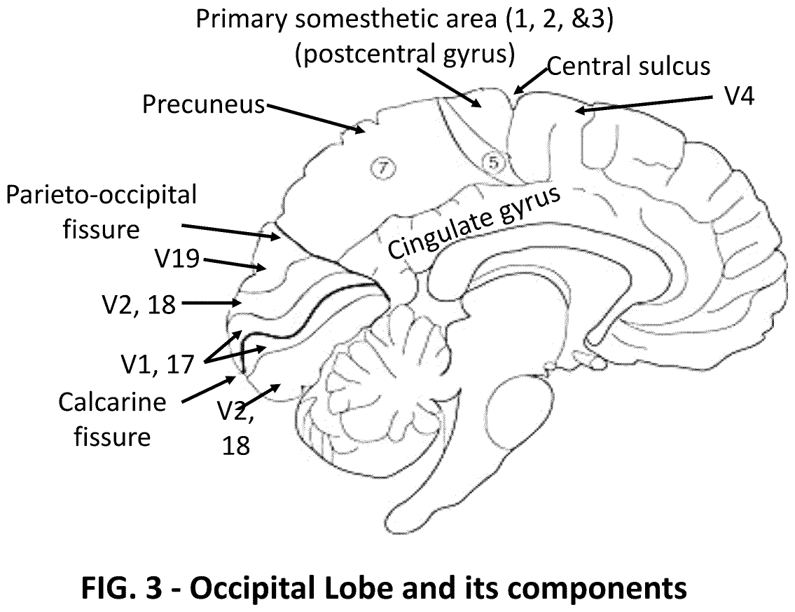

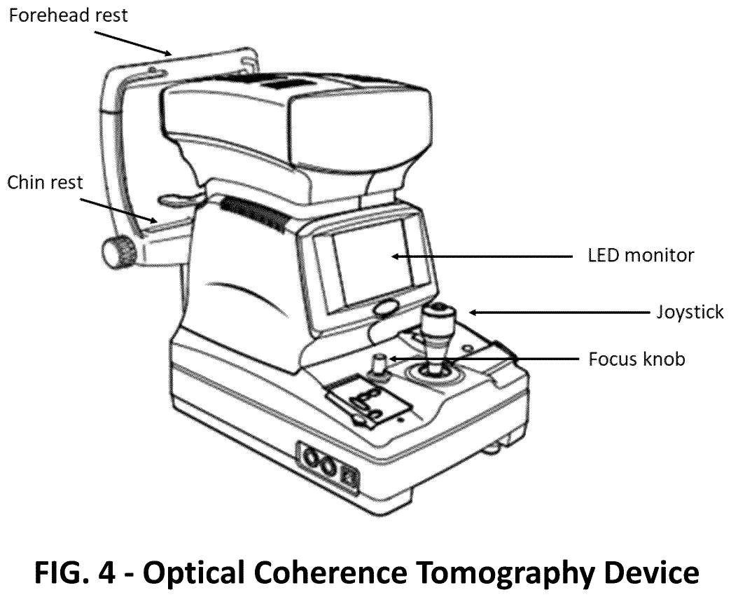

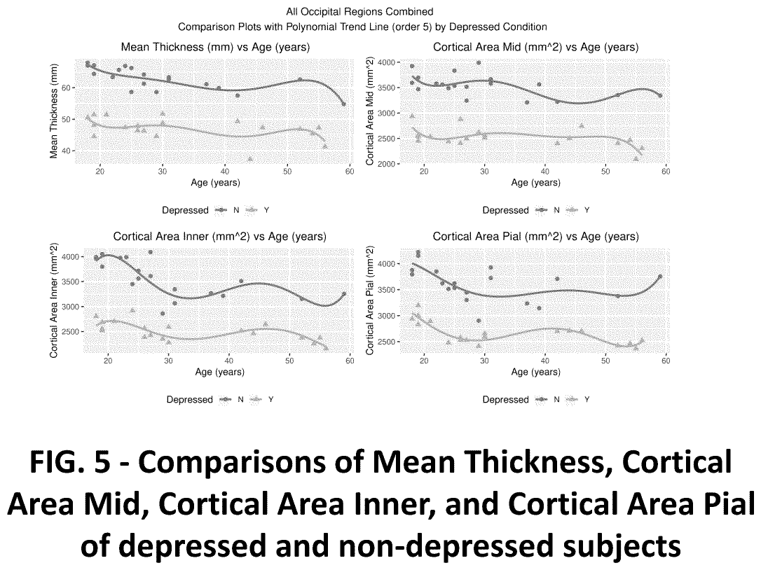

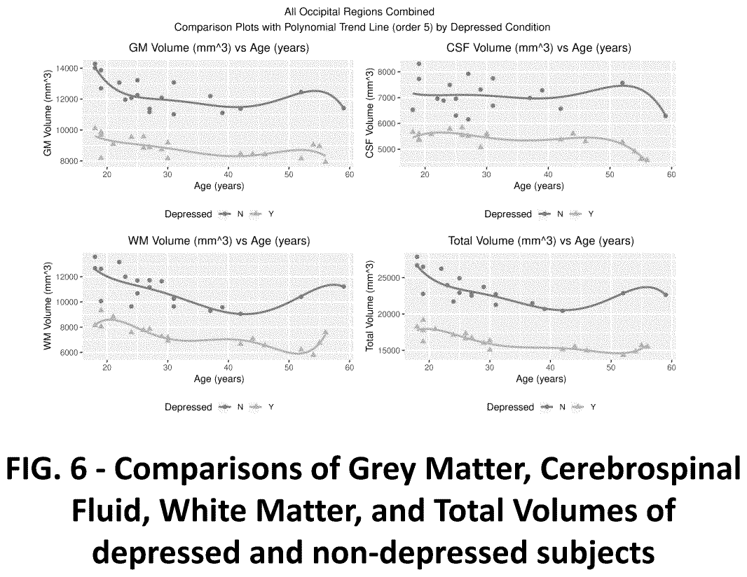

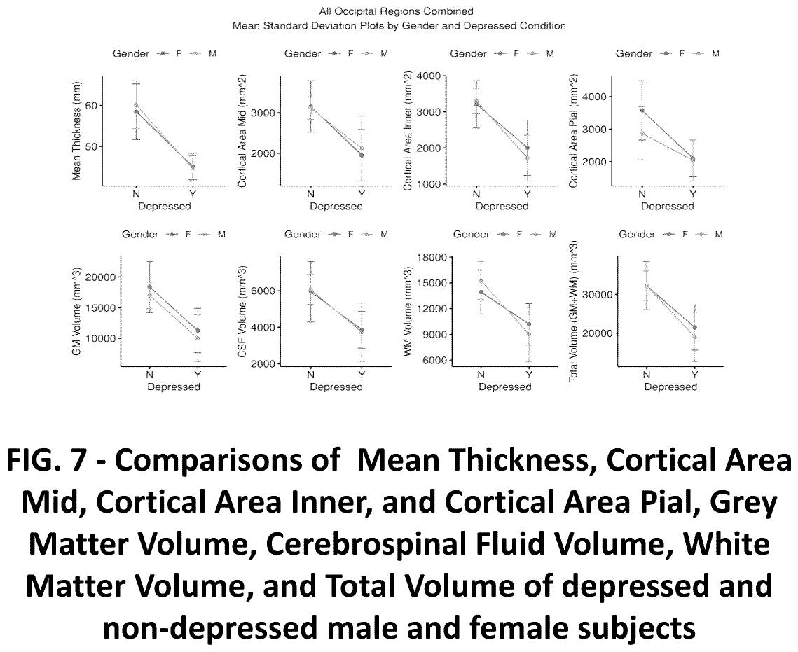

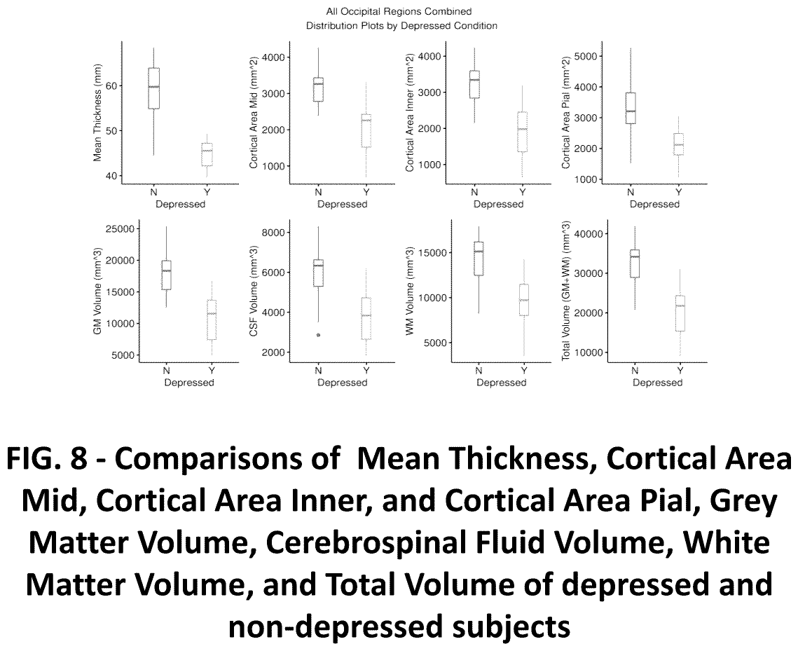

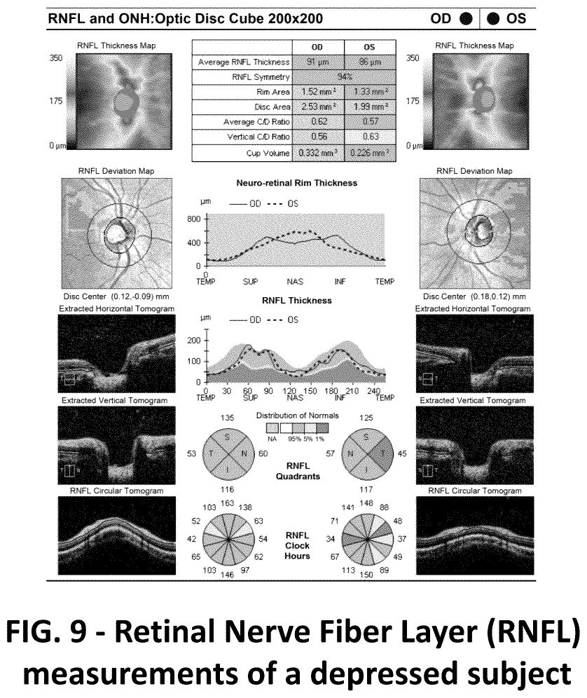

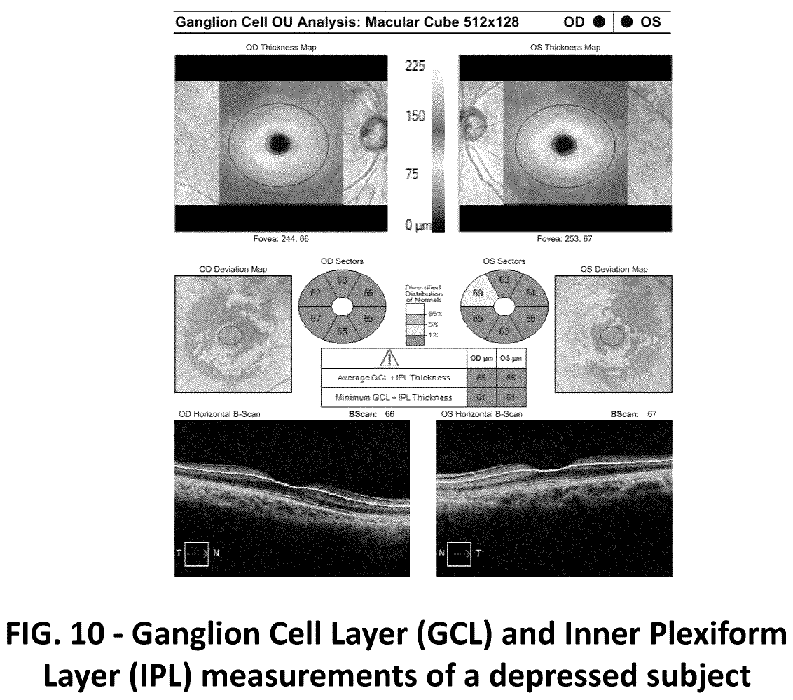

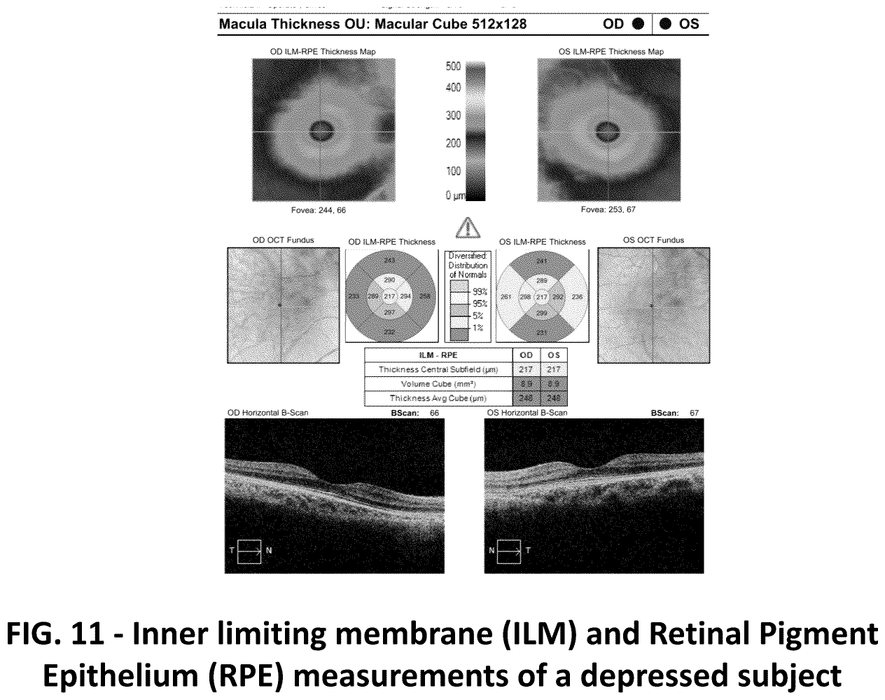

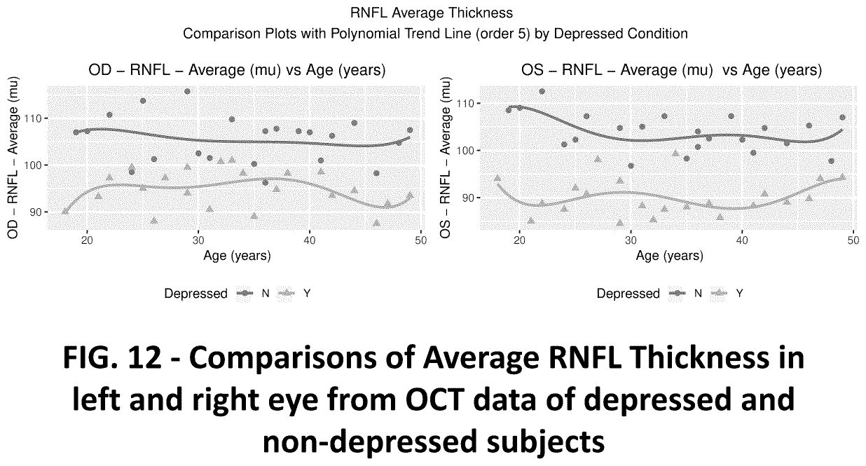

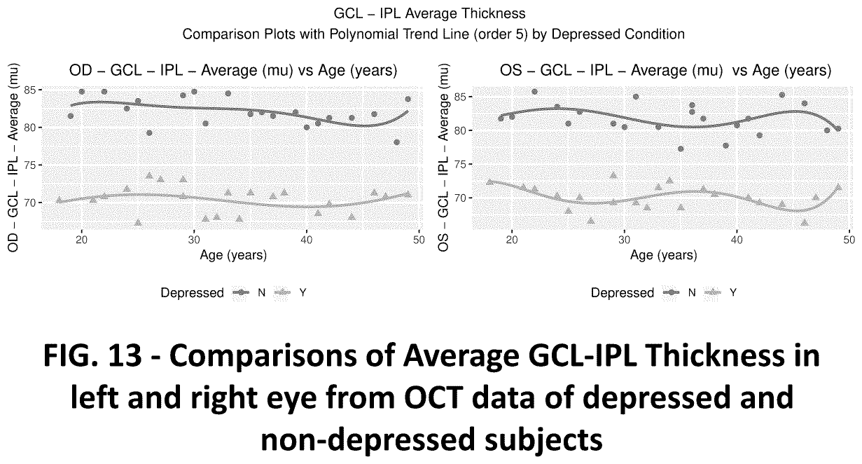

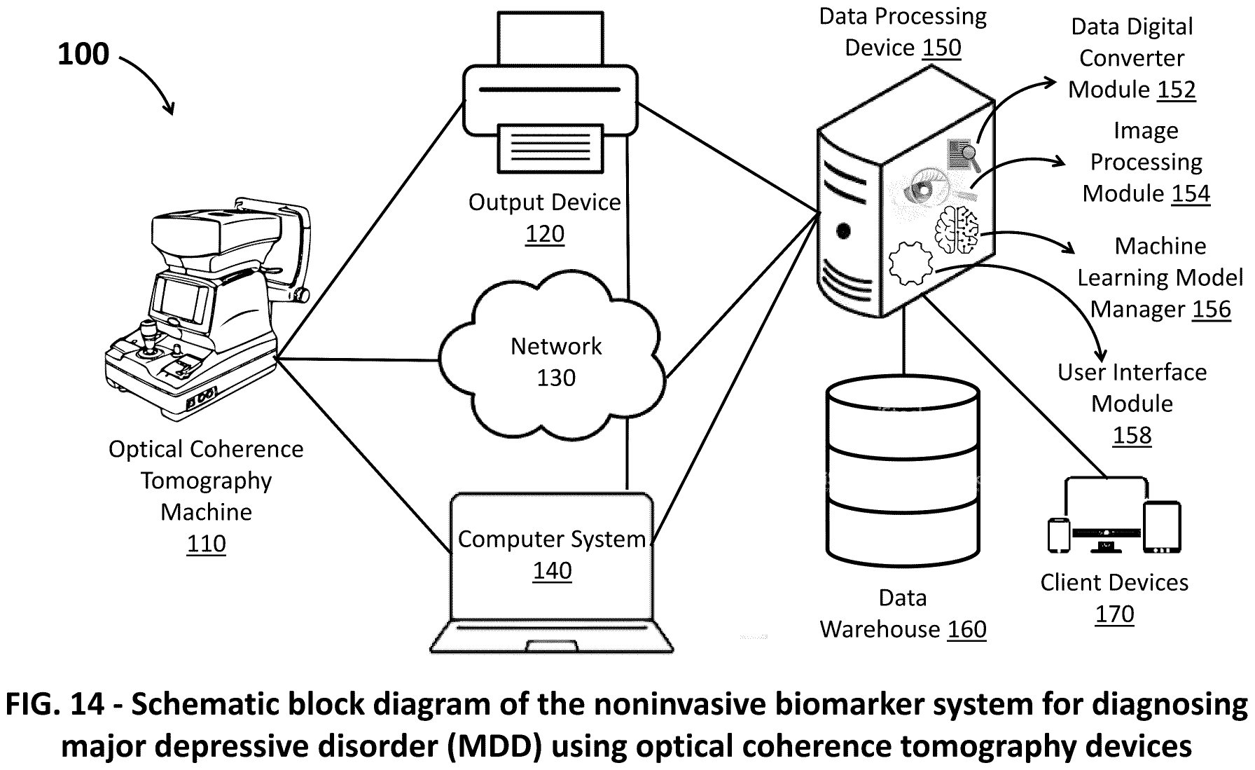

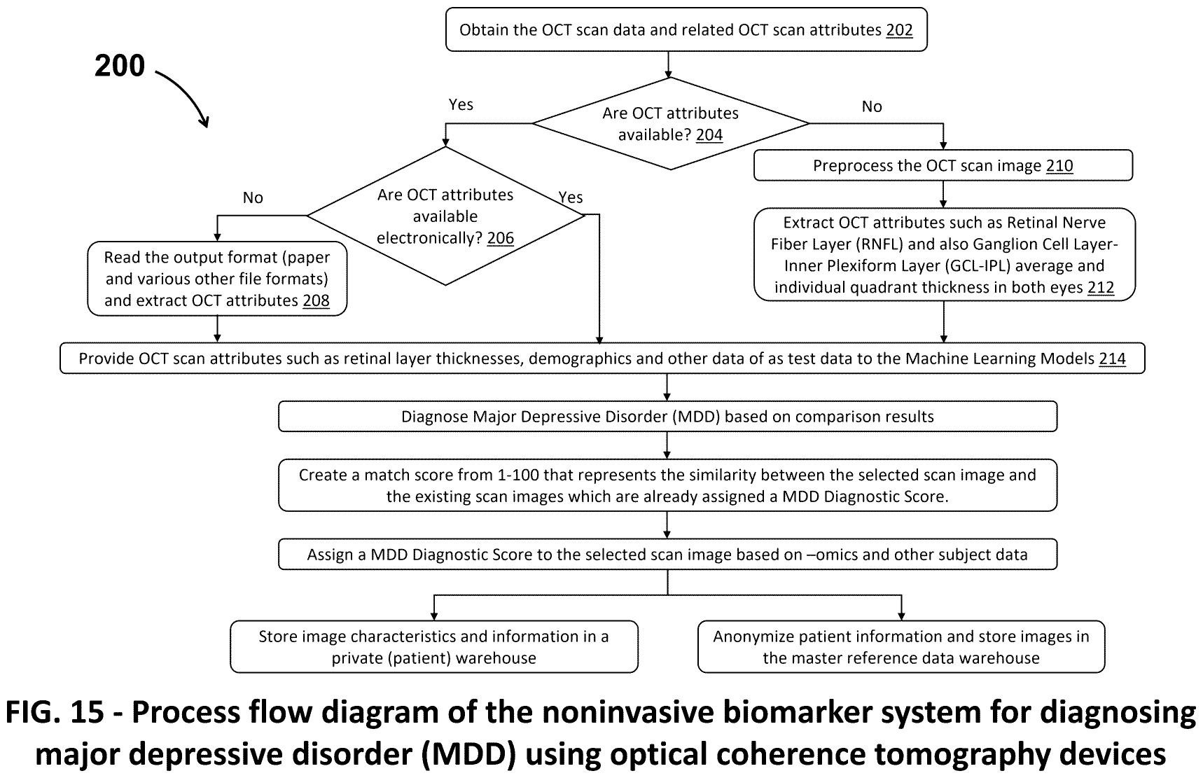

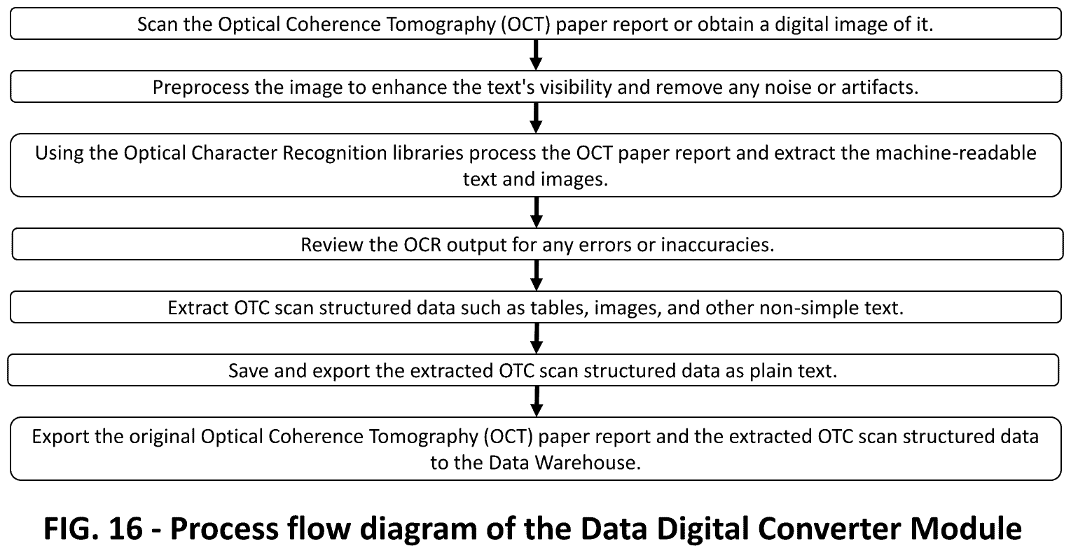

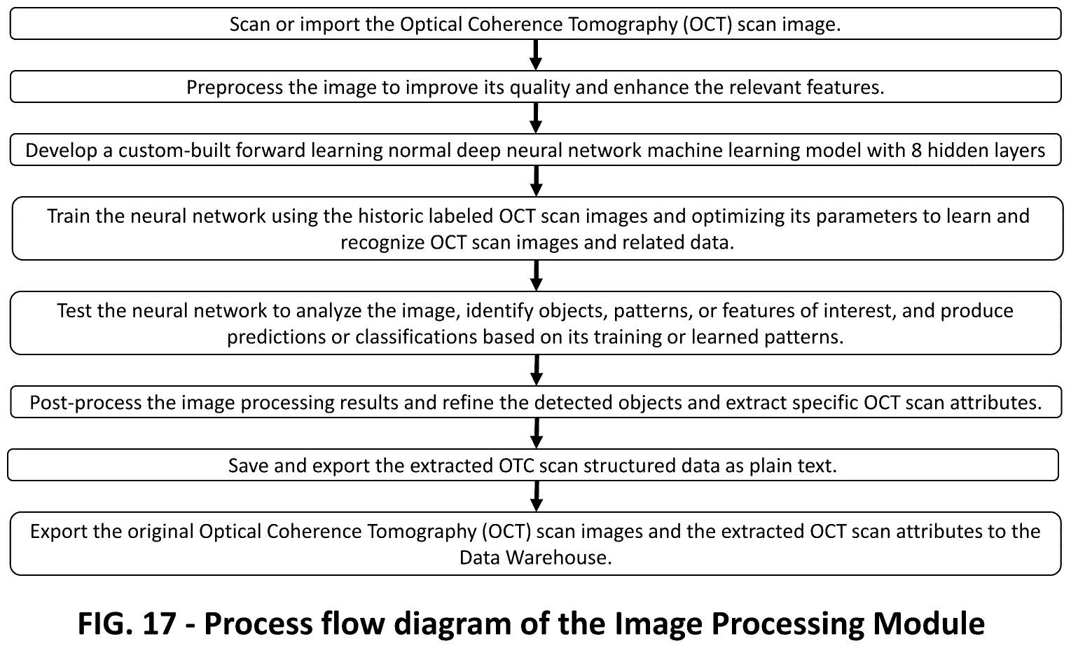

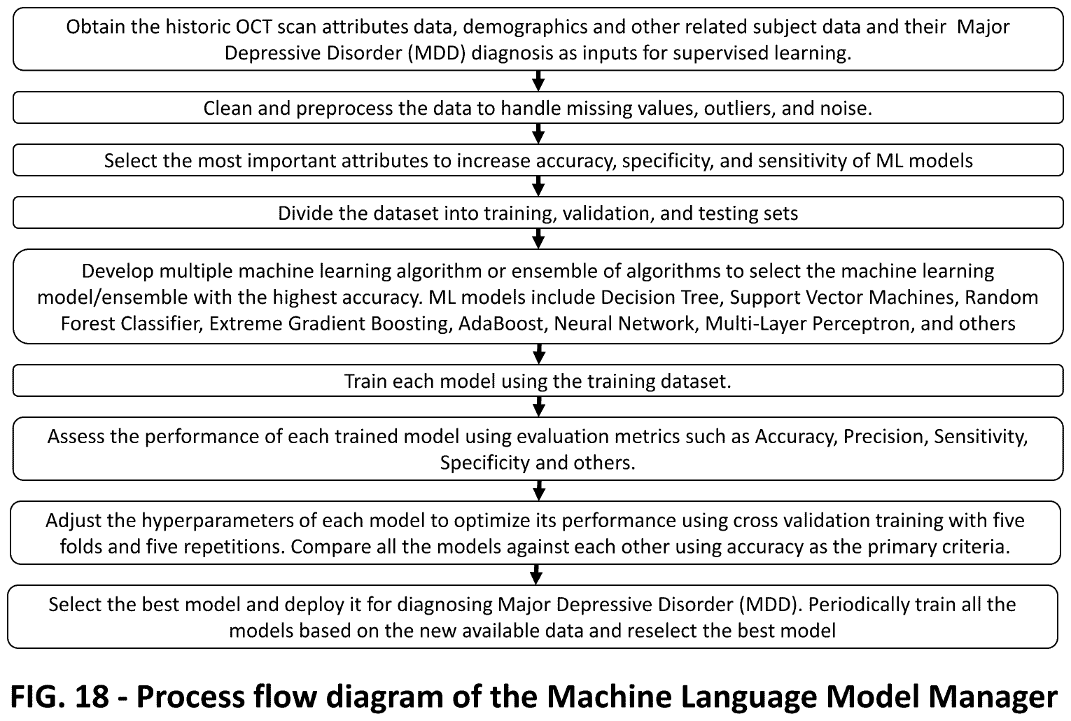

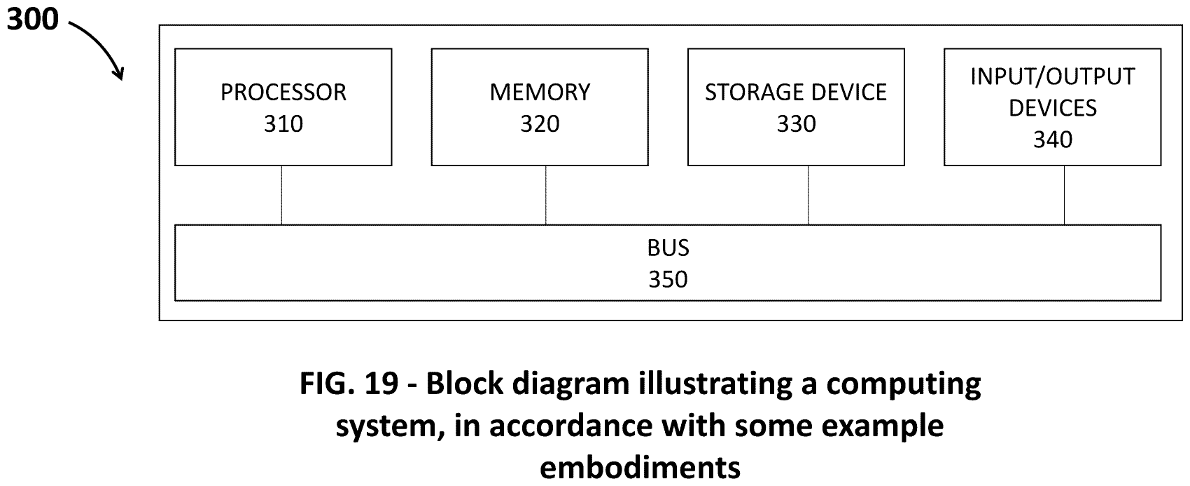

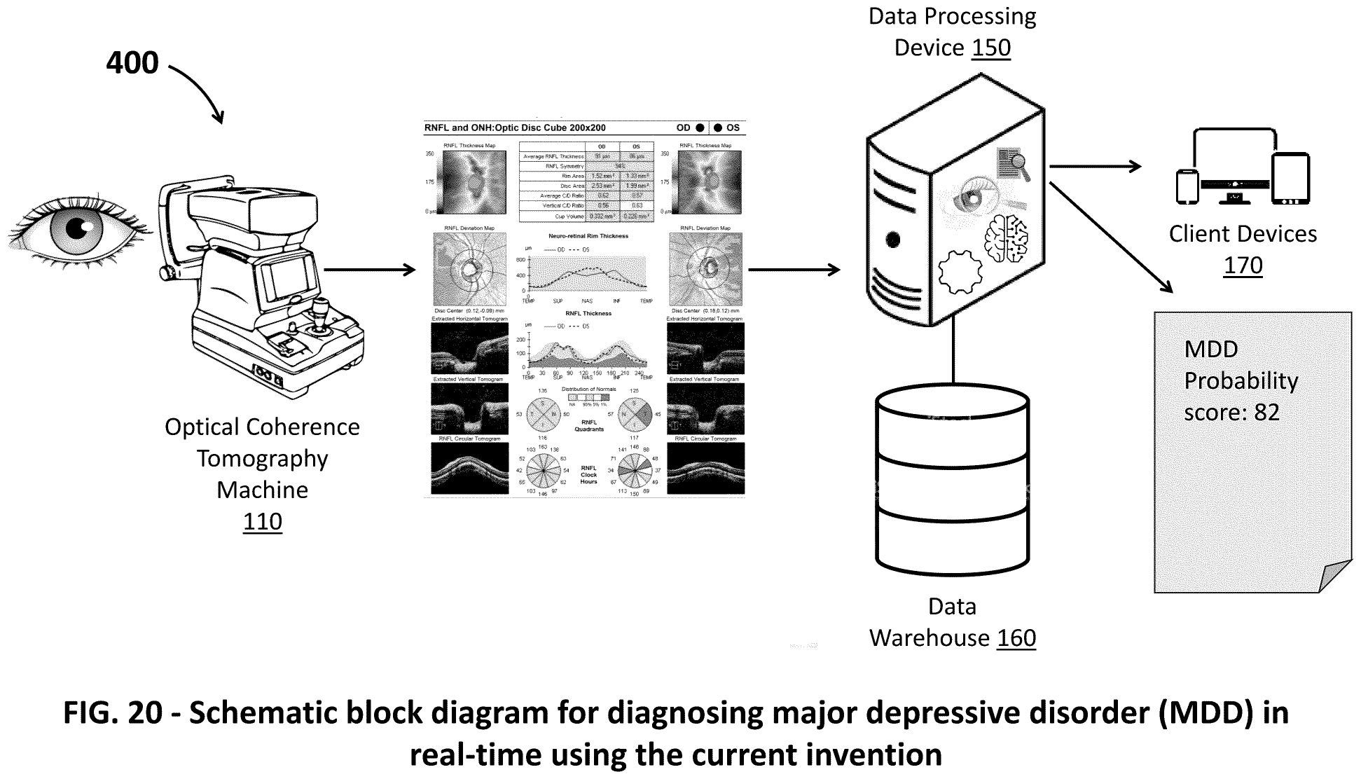

The Occipital Lobe (FIG. 3) is one of the four primary lobes of the cerebral cortex, which refers to the outer layer of the brain. It is located at the posterior of the brain and is responsible for receiving and processing visual stimuli. It also helps us interpret these stimuli through various means, such as color, motion, depth, and shape.

These are the key features and their purpose within the Occipital Lobe:

Primary Visual Cortex (V1): The primary visual cortex is the first region in the brain to receive and interpret visual information that is taken in by the eyes. It plays a role in basic processing, such as detecting edges, orientation, and contrast.

Visual Association Areas: Beyond V1, the Occipital Lobe contains additional visual association areas, such as V2, V3, V4, and V5. These association areas are responsible for visual processing at a higher level, such as recognizing objects, perceiving color, detecting motion, and spatial awareness.

Depth and Motion Perception: The Occipital Lobe, particularly the additional visual association areas and V5, is very important for depth perception, motion perception, and the interpretation of these phenomena.

Visual Imagery and Memory: The Occipital Lobe is involved in generating and processing mental images, which helps our memory by recognizing visual stimuli.

Integration with Other Brain Regions: The Occipital Lobe communicates with other brain regions to integrate visual information and promote cognition and perception. It receives information from the thalamus and connects with other lobes of the cerebral cortex to support our intake of stimuli.

Depression mainly impacts the emotional, behavioral, and cognitive aspects of mental health. While it may not target particular brain areas, it has indirect effects on functioning that may be due to impacts in certain regions of the brain. Here are a few ways in which depression can influence the Occipital Lobe:

Altered Brain Activation: Studies with neuroimaging techniques have demonstrated that individuals with depression may show altered patterns of brain activation during visual processing tasks. This suggests that there may be changes in the neural processing of visual information.

Visual Perception Changes: Depression can impact the way individuals interpret visual stimuli and may cause disturbances such as changes in color perception, heightened response to light, and blurriness. These changes may be associated with alterations in the Occipital Lobe or its interactions with other brain regions.

Cognitive Impairments: Depression has been shown to be linked with cognitive impairments in attention, concentration, and memory. The Occipital Lobe plays a role in visual attention and memory, and disrupting its function can contribute to these cognitive impairments.

Sleep and Circadian Rhythm Disturbances: Depression is often correlated with disturbances in sleep patterns and circadian rhythms. These changes are linked to the Occipital Lobe, which plays a role in mediating sleep-wake cycles and processing light and darkness.

### DESCRIPTION OF THE INVENTION

The following detailed description is directed to detecting depression in a subject based on the analysis of the Retinal Nerve Fiber Layer (RNFL), Ganglion Cell Layer (GCL), Inner Plexiform Layer (IPL), and other retinal layer thicknesses in each eye in all four (temporal, superior, nasal, and inferior) quadrants and the average thickness of each of these retinal layers using measurements obtained from optical coherence tomography image data. In the following detailed description, reference is made to the accompanying drawings, which form a part hereof and which are shown by way of illustration embodiments that can be practiced. It is to be understood that other embodiments can be utilized, and structural or logical changes can be made without departing from the scope. Therefore, the following detailed description is not to be taken in a limiting sense, and the scope of embodiments is defined by the appended claims and their equivalents.

Various operations can be described as multiple discrete operations, in turn, in a manner that can be helpful in understanding embodiments; however, the order of description should not be construed to imply that these operations are order dependent. The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure information of a sample can be obtained using OCT imaging based on the detection of spectral interference. Such imaging can be two-dimensional (2-D) or three-dimensional (3-D), depending on the application. Structural imaging can be of an extended depth range relative to prior art methods and can be performed in real-time. OCT imaging using various devices and formats can be used for producing 2-D or 3-D images.

Unless otherwise noted or explained, all technical and scientific terms used herein are used according to conventional usage and have the same meaning as commonly understood by persons of ordinary skill in the art to which the disclosure belongs. Although exact materials, apparatuses, systems, methods, techniques, or equivalent or similar to those described above can be used in the testing or practice of the present disclosure, an example set of suitable apparatuses, systems, methods, and techniques are described below.

All publications, patent applications, patents, and other references mentioned herein are incorporated by reference in their entirety. In case of conflict, the present specification, including an explanation of terms, will control. In addition, the methods, systems, apparatuses, materials, and examples are illustrative only and not intended to be limiting.

A study was conducted to identify the effects of Major Depressive Disorder (MDD) on the brain and how these effects are propagated throughout the visual system.

### Effect of Major Depressive Disorder (MDD) in all Occipital Regions of Interest (ROI) in the Brain:

To determine the effect of Major Depressive Disorder (MDD) in all Occipital Regions of Interest (ROI) of the brain, fMRI images of 19 subjects who are non-depressed and 19 subjects who are unmedicated and currently experiencing Major Depressive Disorder are analyzed. Non-depressed control participants with no history of depression or other psychiatric disorders include eight (8) males (Range_{AGE}=18-52; Mean_{AGE}=31.625) and eleven (11) females (Range_{AGE}=18-59; Mean_{AGE}=28.545). MDD subjects were all unmedicated, experiencing a current depressive episode at the time of scanning, which was determined by screening for research purposes using the SCID-I/NP. Participants include: 8 males (Range_{AGE}=19-56; Mean_{AGE}=39.250) and 11 females (Range_{AGE}=18-52; Mean_{AGE}=29.364).

An analysis is performed in all Regions Of Interest (ROI) in the brain and Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume is computed for each subject.

To determine the effect of MDD on various regions of interest (ROI) in the brain, statistical analysis was performed on control and MDD subjects' measures: Mean Thickness (mm), Grey Matter Volume (mm3), Cerebrospinal Fluid Volume (mm3), White Matter Volume (mm3), Total Volume (GM+WM) (mm3), Cortical Area Mid (mm2), Cortical Area Inner (mm2), and Cortical Area Pial (mm2), in each ROI.

As shown in FIG. 5, FIG. 6, and FIG. 8, MDD showed a statistically significant effect on all the ROI attributes for all of the combined occipital regions.

As shown in FIG. 7, MDD showed a statistically significant effect on all the ROI attributes for all of the combined occipital regions in both males and females.

However, periodic functional Magnetic Resonance Imaging (fMRI) scans may be difficult to obtain due to limited access to scanners, especially in certain geographic regions or hospitals. With the high demand for fMRI scans for other medical conditions, patients may experience long wait times for appointments fMRI scans are also very expensive, resulting in many individuals being unable to afford the imaging. In certain cases, insurance may not cover the scan, and the costs vary based on the healthcare system and location. A scan generally takes a significant amount of time to complete and involves lying in a confined space inside the scanner, remaining still for 30 minutes to 1 hour, and following other directions. For individuals who are claustrophobic or have challenges remaining still, this procedure may cause discomfort. The scanner may also produce loud noises while imaging, which may be distressing for some individuals, even with the provided ear protection. Before undergoing the scan, there are certain necessary preparations, such as removing metal objects, wearing certain clothes, or fasting for the day, based on the specific requirements of the scan. These preparations may require additional planning, which may be inconvenient. Additionally, some medical conditions and implants, such as cochlear implants, metal fragments, and pacemakers, may prevent individuals from undergoing an fMRI scan.

OCT technology is widely available in ophthalmology clinics and vision centers and provides exceptional high-resolution images of the retina. This allows for detailed visualization and detection of subtle changes in the layers and structures of the retina. This makes it valuable for diagnosing and monitoring conditions, as the retinal layers are associated and connected to the Occipital Lobe of the brain. The thickness of various retinal layers can be compared between depressed and non-depressed patients.

To determine the effect of MDD on the retinal layers, statistical analysis was performed on both RNFL and GCL-IPL layers for all control and MDD subject measures in both eyes (Left Eye—OS—Oculus Sinister and right eye—OD—oculus dextrus) using average and individual quadrant measures: OD—Temporal (μm), OD—Superior (μm), OD—Nasal (μm), OD—Inferior (μm), OD—Average (μm), OS—Temporal (μm), OS—Superior (μm), OS—Nasal (μm), OS—Inferior (μm), and OS—Average (μm).

As shown in FIG. 12 and FIG. 13, MDD showed a statistically significant effect on RNFL thickness in all the quadrants and also on the average RNFL thickness. In addition, MDD also showed a statistically significant effect on GCL-IPL thickness in all the quadrants and also on the average GCL-IPL thickness.

### DETAILED DESCRIPTION OF THE INVENTION

Optical coherence tomography (OCT) is a non-contact, noninvasive imaging modality for high-resolution, depth-resolved, cross-sectional, and three-dimensional (3D) imaging of biological tissue. Among its many applications, OCT has found widespread clinical use in ocular imaging and is routinely performed in the office of the ophthalmologist/optometrist very quickly and easily with minimal expertise during the annual checkup.

As an example, currently, OCT image data is most commonly used to measure the retinal nerve fiber layer changes and ganglion cell complex loss to assist in detecting and diagnosing optic neuropathic diseases such as glaucoma.

While the present invention may be embodied in many different forms, several specific embodiments are discussed herein with the understanding that the present disclosure is to be considered only as an exemplification of the principles of the invention, and it is not intended to limit the invention to the embodiments illustrated.

It is an object of the present invention to provide methods for early diagnosis and treatment of Major Depressive Disorder (MDD) in a subject. In one embodiment of the present invention, as shown in FIG. 20, a method for determining MDD status in a subject is provided, the method comprising the steps of (a) imaging both eyes of the individual using the OCT device; (b) passing information about OCT scan data and related attributes of the eyes to a server containing machine language models; (c) the server equipped with machine language models is capable of retrieving and updating the data warehouse, which is comprised of anonymized data of various subjects including subject-specific data such as demographic data (age, gender, ethnicity, and others), medical history (height, weight, body mass index, body fat, medical conditions), stress information, nutrition and exercise data, prescription history, occupation, and more; (d) assessing and determining the likelihood of MDD using machine language models; and (e) providing a probability score of MDD to the subject with related MDD data from other subjects (justification).

The data processing system 100 processes the retinal layer thicknesses to diagnose Major Depressive Disorder (MDD).

FIG. 14 illustrates the Data Processing System 100 used in the diagnosis of Major Depressive Disorder (MDD) using Optical Coherence Tomography (OCT) scan data and related OCT scan attributes. More specifically, the analysis processing system 100 predicts the probability of Major Depressive Disorder (MDD) in humans based on the Retinal Nerve Fiber Layer (RNFL) and also Ganglion Cell Layer-Inner Plexiform Layer (GCL-IPL) thickness measured in both eyes (Left Eye—OS—Oculus Sinister and right eye—OD—oculus dextrus) using average and individual quadrant measures: OD—Temporal (μm), OD—Superior (μm), OD—Nasal (μm), OD—Inferior (μm), OD—Average (μm), OS—Temporal (μm), OS—Superior (μm), OS—Nasal (μm), OS—Inferior (μm), and OS—Average (μm) using commercial or experimental OCT/OCTA devices 110.

According to one aspect of the invention, automated methods are then executed using these measurements to identify Major Depressive Disorder (MDD). The analysis processing system 100 determines or predicts the probability of Major Depressive Disorder (MDD) using Data Processing Device 150 containing Machine Learning Model Manager 156, subsequently described in detail in FIG. 18. The analysis processing system 100 is configured to enable medical service providers to tailor the prediction of the probability of Major Depressive Disorder (MDD) to individual patients depending on their respective characteristics, such as demographic data (age, gender, ethnicity, and others), medical history (height, weight, body mass index, body fat, medical conditions), stress information, nutrition and exercise data, prescription history, occupation, and more, usually stored in the Data Warehouse 160 on the Data Processing Device 150 in along with the Machine Learning Model Manager 156.

In one embodiment, Optical Coherence Tomography systems or similar devices 110 can be connected to an Output Device 120, which is capable of printing or generating OCT scan data and related OCT attributes in various formats. This Output Device 120 can then be connected to the Data Processing Device 150 using direct cables or over the Network 130 (e.g., a wired or wireless communications network) or in any other fashion. Data Processing Device 150 contains a Data Digital Converter Module 152, subsequently described in detail in FIG. 16. Data Digital Converter Module 152 is capable of reading the OCT scan data and related OCT attributes in paper and various other file formats, extract OCT attributes, and converting them to a digital output.

In another embodiment, Optical Coherence Tomography systems or similar devices 110 that are capable of storing OCT scan data and related OCT attributes in digital format can be directly connected over Network 130 (e.g., a wired or wireless communications network) to a Data Processing Device 150.

In another embodiment, Optical Coherence Tomography systems or similar devices 110, which can store OCT images in their memory, file storage system, or external systems can be connected using cables or tightly coupled or integrated to a Computer System 140, which is capable of replicating the OCT images. This Computer System 140 can then be connected to the Data Processing Device 150 using direct cables or over the Network 130 (e.g., a wired or wireless communications network) or in any other fashion. Data Processing Device 150 contains an Image Processing Module 154, subsequently described in detail in FIG. 17. Image Processing Module 154 can preprocess images from the OCT scan, extract the relevant OCT attributes, and convert them into a digital output.

The Data Processing Device 150 is configured to obtain OCT scan images and related OCT attributes, such as various measures of Retinal Nerve Fiber Layer (RNFL) and Ganglion Cell Layer-Inner Plexiform Layer (GCL-IPL) thicknesses and other measures. The Data Processing Device 150 is generally a computing system configured to host at least one or more Machine Learning Model Managers 156. The data processing device 150 is configured to perform the processing operations for diagnosing Major Depressive Disorder (MDD), including data scanning functions, image processing functions, scoring functions, machine learning model calculations, and user interface functions which are subsequently described.

For example, the Data Processing Device 150 can be combined with the Data Warehouse 160 for storing OCT scan data, OCT scan images, related OCT scan attributes data, and also demographic data (age, gender, ethnicity, and others), medical history (height, weight, body mass index, body fat, medical conditions), stress information, nutrition and exercise data, prescription history, occupation, and more of the subjects which can be used as training data for the Machine Learning Model Manager 156.

In another example, the Data Processing Device 150 is connected with the Client Devices 170 over the Network (Internet, Extranet, or a VPN connection) to provide a user interface enabling subjects, end users, and related parties to interact with the Data Processing Device 150 directly. The process flow diagram of the Machine Learning Model Manager 156 is further described in FIG. 18.

The Machine Learning Model Manager 156 and scoring module of the Data Processing Device 160 are configured to incorporate relationships between the OCT scan attributes data and Major Depressive Disorder (MDD). One or more Machine Learning Model Managers 156 are trained, and Accuracy, Precision, Sensitivity, Specificity, and other performance measures are computed using the OCT scan attributes data such as various retinal layer thicknesses and MDD indicator of each of the subjects, demographic data (age, gender, ethnicity, and others), medical history (height, weight, body mass index, body fat, medical conditions), stress information, nutrition and exercise data, prescription history, occupation, and more.

For diagnosing MDD, Machine Learning Model Manager 156 uses various retinal layer thicknesses, demographic data (age, gender, ethnicity, and others), medical history (height, weight, body mass index, body fat, medical conditions), stress information, nutrition and exercise data, prescription history, occupation, and more of the current subject as test data to the Machine Learning Models and provides a probability score for the Major Depressive Disorder (MDD) including the closely related data from other subjects from the Data Warehouse 160.

FIG. 15 shows a flow diagram of an example process for diagnosing Major Depressive Disorder (MDD) in a subject by data processing system 100 of FIG. 14. Process 200 includes obtaining the OCT scan data and extracting related OCT scan attributes 202 of a subject during a routine OCT imaging that is conducted at the doctor's office. OTC scan data may only include images of both eyes or images of both eyes with related attribute data. The output format may be electronic, paper, or other related formats (FIG. 9, FIG. 10, and FIG. 11).

In one aspect, processing the OCT scan data can include preprocessing the output formats (paper and various other file formats) of the OCT scans with a Data Digital Converter Module 152 in the Data Processing Device 150 of FIG. 1 to extract OCT scan attributes such as Retinal Nerve Fiber Layer (RNFL) and also Ganglion Cell Layer-Inner Plexiform Layer (GCL-IPL) measurements in both eyes (Left Eye-OS-Oculus Sinister and right eye-OD-oculus dextrus) using average and individual quadrant thickness measures: OD—Temporal (μm), OD—Superior (μ m), OD—Nasal (μm), OD—Inferior (μm), OD—Average (μm), OS—Temporal (μm), OS—Superior (μm), OS—Nasal (μm), OS—Inferior (μm), and OS—Average (μm). These OCT scan attributes are then provided as input data by the Machine Learning Model Manager 156 of FIG. 1 for various machine learning models to diagnose Major Depressive Disorder (MDD).

In another aspect, electronically available OCT scan attributes such as Retinal Nerve Fiber Layer (RNFL) and also Ganglion Cell Layer-Inner Plexiform Layer (GCL-IPL) measurements in both eyes (Left Eye—OS—Oculus Sinister and right eye—OD—oculus dextrus) using average and individual quadrant thickness measures: OD—Temporal (μm), OD—Superior (μm), OD—Nasal (μm), OD—Inferior (μm), OD—Average (μm), OS—Temporal (μm), OS—Superior (μm), OS—Nasal (μm), OS—Inferior (μm), and OS—Average (μm) can be sent directly to the Data Processing Device 150 without any preprocessing. These OCT scan attributes are then provided as input data by the Machine Learning Model Manager 156 of FIG. 1 for various machine learning models to diagnose Major Depressive Disorder (MDD).

In another aspect, processing the OCT scan data can include preprocessing the OCT image with an Image Processing Module 154 in the Data Processing Device 150 to extract OCT attributes such as Retinal Nerve Fiber Layer (RNFL) and also Ganglion Cell Layer-Inner Plexiform Layer (GCL-IPL) measurements in both eyes (Left Eye-OS-Oculus Sinister and right eye-OD-oculus dextrus) using average and individual quadrant thickness measures: OD—Temporal (μm), OD—Superior (μm), OD—Nasal (μm), OD—Inferior (μm), OD—Average (μm), OS—Temporal (μm), OS—Superior (μm), OS—Nasal (μm), OS—Inferior (μm), and OS—Average (μm). These OCT scan attributes are then provided as input data by the Machine Learning Model Manager 156 of FIG. 1 for various machine learning models to diagnose Major Depressive Disorder (MDD).

The specific type of machine learning model can be one of a variety of models based on Accuracy, Precision, Sensitivity, Specificity, and other measures based on the demographic data (age, gender, ethnicity, and others), medical history (height, weight, body mass index, body fat, medical conditions), stress information, nutrition and exercise data, prescription history, occupation, and more.

1. A method for predicting and diagnosing the occurrence of major depressive disorder (MDD) in a human subject, the method comprising:
(a) obtaining a plurality of features of various Retinal Layer thickness measurements using Optical Coherence Tomography (OCT) devices; and
(b) selecting a combination of features from the plurality of features of various Retinal Layer thickness measurements based on biological analysis, traditional statistical analysis, and machine learning dimension reduction analysis; and
(c) selecting a combination of features from the plurality of features of various Retinal Layer thickness measurements based on a robustness metric associated with insensitivity to manufacturing variabilities of Optical Coherence Tomography (OCT) devices and a performance metric associated with predicting a Major Depressive Disorder (MDD) classification; and
(d) training one or more machine learning models to predict and diagnose the Major Depressive Disorder (MDD) classification using the combination of features measured by the Optical Coherence Tomography (OCT) devices.

2. The method according to claim 1, the method further comprising:
(a) predicting a specific subtype of Major Depressive Disorder (MDD) such as Melancholic, Atypical, Psychotic, Seasonal Affective Disorder (SAD), and others; and
(b) predicting a risk level of developing Major Depressive Disorder (MDD) such as high risk, low risk, or no risk for developing MDD; and
(c) predicting a probability score and a detailed analysis of the prediction.

3. A method for predicting and diagnosing the occurrence of neurological diseases such as Multiple Sclerosis (MS), Alzheimer's disease, Parkinson's disease, and others in a human subject, the method comprising:
(a) obtaining a plurality of features of various Retinal Layer thickness measurements using Optical Coherence Tomography (OCT) devices; and
(b) selecting a combination of features from the plurality of features of various Retinal Layer thickness measurements based on biological analysis, traditional statistical analysis, and machine learning dimension reduction analysis; and
(c) selecting a combination of features from the plurality of features of various Retinal Layer thickness measurements based on a robustness metric associated with insensitivity to manufacturing variabilities of Optical Coherence Tomography (OCT) devices and a performance metric associated with predicting the classification of neurological diseases such as Multiple Sclerosis (MS), Alzheimer's disease, Parkinson's disease, and others; and
(d) training one or more machine learning models to predict and diagnose the classification of neurological diseases such as Multiple Sclerosis (MS), Alzheimer's disease, Parkinson's disease, and others using the combination of features measured by the Optical Coherence Tomography (OCT) devices.

4. The method according to claim 3, the method further comprising:
(a) predicting a risk level of developing neurological diseases like Multiple Sclerosis (MS), Alzheimer's disease, Parkinson's disease, and others such as high risk, low risk, or no risk for developing them; and
(b) predicting a probability score and a detailed analysis of the prediction.

5. A method for predicting and diagnosing the occurrence of major depressive disorder (MDD) in a human subject, the method comprising:
(a) obtaining a plurality of features of various measurements of the gyri, including Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume of the Occipital Lobe using voxel analysis of functional Magnetic Resonance Imaging (fMRI) scans; and
(b) selecting a combination of features from the plurality of features of various measurements of the gyri of the Occipital Lobe based on biological analysis, traditional statistical analysis, and machine learning dimension reduction analysis; and
(c) selecting a combination of features from the plurality of features of various measurements of the gyri of the Occipital Lobe based on a robustness metric associated with insensitivity to manufacturing variabilities of functional Magnetic Resonance Imaging (fMRI) devices and a performance metric associated with predicting a Major Depressive Disorder (MDD) classification; and
(d) training one or more machine learning models to predict and diagnose the Major Depressive Disorder (MDD) classification using the combination of features measured by the functional Magnetic Resonance Imaging (fMRI) devices.

6. The method according to claim 5, the method further comprising:
(a) predicting a specific subtype of Major Depressive Disorder (MDD) such as Melancholic, Atypical, Psychotic, Seasonal Affective Disorder (SAD), and others; and
(b) predicting a risk level of developing Major Depressive Disorder (MDD) such as high risk, low risk, or no risk for developing MDD; and
(c) predicting a probability score and a detailed analysis of the prediction.
